(19) United States
(12) Patent Application Publication
(10) Pub. No.: US 2005/0070767 A1
(43) Pub. Date: Mar. 31, 2005

(54) Patient monitoring system

(76) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 10/952,629

(22) Filed: Sep. 29, 2004

(30) Foreign Application Priority Data
Sep. 29, 2003 (DE) 10345171.4

(51) Int. Cl. A61B 5/00

(52) U.S. Cl. 600/300; 128/903

(57) ABSTRACT

The invention relates to a patient monitoring system which comprises a measuring device disposed in a recording zone and used for recording the patient's medical and diagnostic values and/or for recording the status of a piece of medical apparatus, a transmitter unit connected to the measuring device, a receiver unit communicating wirelessly with said transmitter unit, and an evaluation unit for evaluating data captured using the measuring device, with the evaluation unit being disposed in the recording zone and provided for selection by the receiver unit as well as for initiating the measurement to be performed by means of the measuring device.

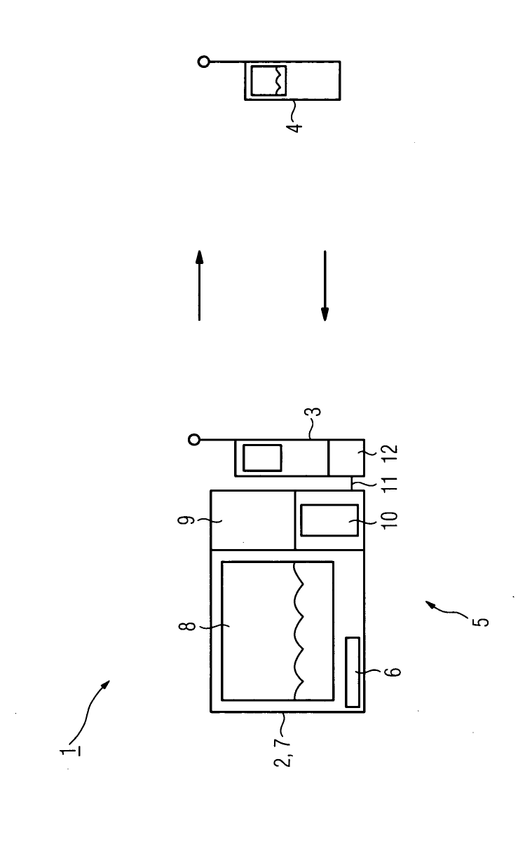

### CROSS REFERENCE TO RELATED APPLICATION

This application claims priority of the German application No. 10345171.4 DE filed Sep. 29, 2003, which is incorporated by reference herein in its entirety.

### FIELD OF THE INVENTION

The invention relates to a patient monitoring system which comprises a measuring device for recording medical and diagnostic values of the patient and/or for recording the status of a piece of medical equipment, a transmitter unit connected to the measuring device, a receiver unit communicating wirelessly with said transmitter unit, and an evaluation unit.

### BACKGROUND OF THE INVENTION

A monitoring system of said kind is known for example from DE 101 56 167 A1.

Equipment known as patient monitors is frequently used for monitoring patients, particularly in intensive care units. Said patient monitors are typically networked with a monitoring center to which alarm signals can be transmitted, usually by means of a hard-wired connection. Equally, forwarding of the signals by wireless means is also possible, as is known in principle from DE 101 56 167 A1. With such equipment, the data forwarded from a measuring device provided for patient monitoring is evaluated in a data processing system in order to check the received measured values for critical values. If necessary, corresponding messages generated by the data processing system can be sent back to the patient. Individual measurements for monitoring the patient are typically carried out at predetermined time intervals.

### SUMMARY OF THE INVENTION

The object of the invention is to specify a patient monitoring system which allows both a particularly reliable recording of the patient's medical and diagnostic values and/or of statuses of a piece of medical apparatus as well as particularly flexible use.

This object is achieved according to the invention by a monitoring system having the features recited the claims. Said monitoring system comprises a measuring device disposed in what is called a recording zone which is not necessarily structurally self-contained, said measuring device serving to record the medical or diagnostic values of the patient, for example the patient's blood pressure, and/or recording a status of a piece of medical apparatus, for example a respirator or an infusion pump. Data captured by means of the measuring device is routed to a transmitter unit which communicates wirelessly with a receiver unit disposed outside of the recording zone. An evaluation unit which is preferably linked to the measuring device as well as to the transmitter unit by means of a hard-wired connection and is disposed outside of the recording zone is provided for the purpose of evaluating and/or storing data recorded using the measuring device. The evaluation unit is linked for data communication purposes to the receiver unit via the bidirectional wireless connection between the transmitter unit and the receiver unit in such a way that the operator of the receiver unit, in particular the doctor, can set the measuring process to be performed using the measuring device in motion at any time from the receiver unit. In addition, it can also be provided that measuring processes are initiated by the patient or automatically at specified time intervals.

The monitoring system is suitable for use both in clinics and for monitoring patients in their private environment. Especially in the last-mentioned case it is of particular advantage in respect of reliable operation of the monitoring system that the evaluation unit is not disposed on the receiver unit side, i.e. generally in the clinic or in the doctor's surgery, but on the measuring device side, i.e. in the vicinity of the patient. If a warning message is generated by the evaluation unit, said message is routed wirelessly, in particular by radio, to the receiver unit. If an immediate warning of the patient is provided at the same time, a corresponding message does not need to be transmitted over a relatively great distance via a radio link or other connection, but can be output immediately by the evaluation unit.

According to a preferred embodiment which offers particular advantages in terms of a compact design and reliable operation, the evaluation unit is integrated into the measuring device. The measuring device is preferably also provided for the graphical presentation of the measured values obtained. In a manner which is customary per se, the measuring device also enables data to be stored.

The data transmission between the transmitter unit and the receiver unit preferably takes place via an existing mobile radio network providing virtually total coverage. In the process the transmitted data is preferably anonymized or encrypted. According to a preferred embodiment, the measuring device is coupled to a telephone module or has a telephone module which allows the alternative dialing of a plurality of receiver units, i.e. usually mobile telephones. If the establishment of a connection to a receiver unit is not possible, the transmitter unit dials one or more receiver units in a predetermined sequence.

Irrespective of the number of receiver units of the monitoring system, according to a preferred embodiment at least one alarm threshold of the measuring device can be set from the receiver unit. The availability of the connection between transmitter and receiver unit is preferably tested automatically on a recurring basis, for example at intervals of several seconds or minutes, with a corresponding message being generated if a connection fails.

The advantage of the invention is in particular that specialist medical staff can dial in at any time via a data communication link to a measurement and analysis system used for patient monitoring in order to inspect data, start or stop measuring processes and specify or modify measurement parameters, whereby the measurement and analysis system is disposed in a particularly operationally reliable manner in the vicinity of the patient and so is autonomously capable of operation, independently of the data communication link.

### BRIEF DESCRIPTION OF THE DRAWINGS

An exemplary embodiment of the invention will be explained below with reference to a FIGURE which shows a patient monitoring system in a schematic representation.

### DETAILED DESCRIPTION OF THE INVENTION

A radio-based monitoring system 1 comprises a measuring device 2, a transmitter unit 3 coupled by a hard-wired connection thereto, and a receiver unit 4 communicating with said transmitter unit 3 via radio. The measuring device 2 and the transmitter unit 3 are disposed in what is referred to as a recording zone 5, for example in an intensive care unit of a clinic or in the patient's home environment. The terms “transmitter unit” and “receiver unit” are chosen to make a conceptual distinction; both units 3, 4 are suitable, as indicated by arrows, both for transmitting and for receiving signals. The measuring device 2 has a sensor port 6 to which a medical apparatus that is not shown, for example an infusion pump or a respirator, can be connected. In addition or alternatively, the sensor port 6 is provided for receiving the patient's medical and diagnostic values, for example blood pressure, pulse, body temperature, blood sugar, body weight or the like. The data captured via the sensor port 6 is forwarded to an evaluation unit 7 which is an integral part of the measuring device 2 which is also referred to in its entirety as a patient monitor. The data received and possibly processed further can be presented on a monitor 8. The FIGURE symbolically shows the waveform of the arterial blood pressure as an example of a physiological parameter. A control unit 9 (comprising control elements which are not shown in further detail) is provided for controlling the measuring device 2, in particular for setting measurement parameters and also display parameters of the monitor 8.

The measuring device 2 further comprises a telephone module 10 which is connected via a line 11 to a data interface 12 of the transmitter unit 3. A commercially available mobile phone is provided both as the transmitter unit 3 and also as the receiver unit 4. The formats of the data to be transmitted between the transmitter unit 3 and the receiver unit 4 are adapted in each case to the applicable mobile radio standards, for example GSM or UMTS. A possibly necessary data conversion is performed for example by the evaluation unit 7 or the telephone module 10.

The patient's physiological parameters that are to be monitored and/or the device parameters of the medical apparatus connected to the sensor port 6 are compared with selectable limit values by the evaluation unit 7, for example at predetermined time intervals. If the measured values lie outside permissible ranges, the sending of a corresponding message to the receiver unit 4 is initiated by the telephone module 10. For this purpose the telephone module 10 has a data encryption mechanism which in a simple case transmits, for example, a patient number known only to the doctor as the user of the receiver unit 4 instead of the patient's name. Furthermore, any encryption algorithms can be used for coding the data stored in the evaluation unit 7 and/or transmitted between the units 3, 4.

If a radio link cannot be set up from the transmitter unit 3 to the receiver unit 4, a further receiver unit (not shown) is selected by means of the telephone module 10 and the message generated by the evaluation unit 7 is transmitted to that receiver unit. According to an alternative embodiment it can also be provided that messages generated by the evaluation unit 7, in particular warning messages, are sent generally to a plurality of receiver units 4. The availability of the radio links between the transmitter unit 3 and the receiver unit 4 or, as the case may be, the receiver units 4 is monitored automatically virtually continuously. A data processing system, for example in a hospital, can also be provided in place of a receiver unit 4.

The person operating the receiver unit 4, in particular the doctor, can, at any time, transmit a message from the receiver unit 4 to the transmitter unit 3, thereby dialing into the evaluation unit 7. In this way the operator can start a measuring process of the measuring device 2 from the receiver unit 4, by means of which process the patient parameters to be monitored and/or statuses of the medical apparatus connected to the measuring device 2 are selectively interrogated. Similarly, the operator is able to interrogate or set limit values or other parameters of the evaluation unit 7 from the receiver unit 4. It is also possible to inspect the data stored in the evaluation unit 7 over a relatively long period of time, for example several days, from the receiver unit 4 and consequently track in particular the trend of measured patient data. The measuring device 2, the evaluation unit 7 and/or the control unit 9 comprise control units (not shown in more detail) which enable the patient to authorize or prohibit inspection of the data present in the measuring device 2 or in the evaluation unit 7 outside of the recording zone 5.

1-9. (cancelled)

10. A patient monitoring system, comprising:
a measuring device arranged in a recording zone within a hospital to record the patient's medical and diagnostic values and to record the status of a medical apparatus;
a transmitter operatively connected to the measuring device;
a receiver that communicates wirelessly with the transmitter unit; and
an evaluation unit to evaluate data obtained by the measuring device, the evaluation unit arranged in the recording zone and is provided for selection by the receiver and initiates the measurement performed by the measuring device.

11. The monitoring system according to claim 10, wherein the evaluation unit is coupled to the measuring device by a hard-wired connection.

12. The monitoring system according to claim 11, wherein the evaluation unit is integrated into the measuring device.

13. The monitoring system according to claim 10, wherein the measuring device is a patient monitor.

14. The monitoring system according to claim 10, wherein a medical apparatus monitored by the measuring device is a respirator.

15. The monitoring system according to claim 10, wherein the medical apparatus monitored by the measuring device an infusion pump.

16. The monitoring system according to claim 10, wherein a telephone module coupled to the measuring device, the evaluation unit, and the transmitter unit, are adapted for dialing up a plurality of receiver units.

17. The monitoring system according to claim 16, wherein the telephone module allows for automatically recurring testing of the connection to the individual receiver units.

18. The monitoring system according to claim 10, wherein the evaluation unit comprises a plurality of alarm parameters that can be set from the receiver unit.

19. The monitoring system according to claim 10, wherein the measuring device is arranged in the recording zone to record the patient's medical and diagnostic values.

20. The monitoring system according to claim 10, wherein the measuring device is arranged in the recording zone to record the status of the medical apparatus.
